(19) United States
(12) Patent Application Publication
(10) Pub. No.: US 2021/0346366 A1
(43) Pub. Date: Nov. 11, 2021

(54) CH24H INHIBITORS FOR PAIN USE

(71) Applicant: Takeda Pharmaceutical Company Limited, Chuo-ku, Osaka-shi, Osaka (JP)

(72) Inventors: Mahnaz ASGHARNEJAD, Cambridge, MA (US); Dimitrios ARKILO, Cambridge, MA (US); Toshiya NISHI, Cambridge, MA (US)

(73) Assignee: Takeda Pharmaceutical Company Limited, Chuo-ku, Osaka-shi, Osaka (JP)

(21) Appl. No.: 17/277,917

(22) PCT Filed: Sep. 19, 2019
(86) PCT No.: PCT/JP2019/038061

§ 371 Date: Mar. 19, 2021

Related U.S. Application Data
(60) Provisional application No. 62/733,927, filed on Sep. 20, 2018.

(51) Int. Cl. A61K 31/4545; A61P 25/06

(52) U.S. Cl. CPC A61K 31/4545; A61P 25/06

(57) ABSTRACT

The present disclosure relates to methods of treating pain by administration of (4-benzyl-4-hydroxypiperidin-1-yl) (2,4′-bipyridin-3-yl)methanone or a pharmaceutically acceptable salt thereof.

### TECHNICAL FIELD

The present invention relates to (4-benzyl-4-hydroxypiperidin-1-yl) (2,4′-bipyridin-3-yl)methanone or a pharmaceutically acceptable salt thereof for use in treatment of pain.

### BACKGROUND OF THE INVENTION

Complex regional pain syndrome (CRPS) was initially identified by Claude Bernard (1813-1878) when he noted that extreme pain could be associated with abnormalities in the autonomic nervous system. Mitchell (1829-1914) coined the term ‘causalgia’ to describe this constellation occurring in veterans of the American Civil War. The term ‘reflex sympathetic dystrophy’ was used until recently when CRPS was agreed upon. CRPS can be subdivided into 3 types: (1) CRPS-I: previously known as reflex sympathetic dystrophy; (2) CRPS-II, previously known as causalgia and defined as CRPS with clinical and/or electrodiagnostic evidence of nerve damage; and 3) CRPS (not otherwise specified) which only partially meets diagnostic criteria, but no better diagnosis can be discerned. CRPS-I and CRPS-II have similar outcomes and response to pain medication. Autonomic changes are often required for the diagnosis and may distinguish between acute CRPS (‘hot’ limb with edema and red coloration) and chronic (‘cold’ limb with atrophy and blue coloration). Although there is significant involvement of the peripheral nervous system, chronic CRPS is thought to be a ‘brain disease’ with demonstrated alterations in both central nervous system (CNS) function as well as structural changes including alterations in cortical representations in both sensory and motor cortex. CRPS is known as one of the most painful disorders and the risk of suicide is significantly higher in patients with CRPS with one study demonstrating that 75% of patients had a high risk for suicide. Amputation has rarely been considered as an option for treatment. Although the pathophysiology has not been established, overactivity of the N-methyl-D-aspartate (NMDA) receptors are thought to play a role and while no drugs are approved for CRPS, ketamine, a NMDA receptor antagonist, has established efficacy in a randomized control trial. Use of an NMDA antagonist along with morphine, decreased pain and cerebral pain representation consistent with brain involvement in CRPS.

### SUMMARY OF THE INVENTION

Aspects of this disclosure relate to a method of treating pain in a mammal comprising administering an effective amount of a composition comprising (4-benzyl-4-hydroxypiperidin-1-yl) (2,4′-bipyridin-3-yl)methanone or a pharmaceutically acceptable salt thereof to the mammal in need thereof. The pain optionally includes inflammatory pain, neuropathic pain, cancer inflammatory pain, post/perioperative pain, and idiopathic pain which is pain of unknown origin, for example, phantom limb pain.

In some embodiments, the pain is Complex Regional Pain Syndrome (CRPS).

In some embodiments, the pain is migraine.

Neuropathic pain is caused by injury or infection of peripheral sensory nerves. It includes, but is not limited to pain from peripheral nerve trauma, herpes virus infection, diabetes mellitus, causalgia, plexus avulsion, neuroma, limb amputation, and vasculitis.

Neuropathic pain is also caused by nerve damage from chronic alcoholism, human immunodeficiency virus infection, hypothyroidism, uremia, or vitamin deficiencies.

Neuropathic pain includes, but is not limited to pain caused by nerve injury such as, for example, “the pain diabetics suffer from”, burn pain, gout pain, osteoarthritic pain, trigeminal neuralgia pain, acute herpetic and postherpetic pain, causalgia pain, chronic pain, diabetic neuropathic pain, fibromyalgia pain, neuropathic pain associated with diabetic peripheral neuropathy, post herpetic neuralgia, fibromyalgia, neuropathic pain associated with spinal cord injury, Complex Regional Pain Syndrome (CRPS), migraine, cluster headache, labor pain, pruritus, and bone cancer pain.

In addition, aspects of this disclosure relate to a method of treating autoimmune disease, cardiovascular disease, diabetic disease, digestive organ disease, ophthalmologic disease or cancer disease in a mammal comprising administering an effective amount of a composition comprising (4-benzyl-4-hydroxypiperidin-1-yl) (2,4′-bipyridin-3-yl)methanone or a pharmaceutically acceptable salt thereof to the mammal in need thereof.

The autoimmune disease includes rheumatoid arthritis, psoriasis, inflammatory bowel disease (e.g., Crohn's disease, ulcerative colitis etc.), Sjogren's syndrome, Behcet's disease, multiple sclerosis, systemic lupus erythematosus, lupus nephritis, discoid lupus erythematosus, Castleman's disease, ankylopoietic spondylarthritis, polymyositis, dermatomyositis (DM), polyarteritis nodosa (PN), mixed connective tissue disease (MCTD), scleroderma, profundus lupus erythematosus, chronic thyroiditis, Graves' disease, autoimmune gastritis, type I diabetes, autoimmune hemolytic anemia, autoimmune neutropenia, thrombocytopenia, atopic dermatitis, chronic active hepatitis, myasthenia gravis, graft versus host disease, Addison's disease, abnormal immunoresponse, arthritis, dermatitis, radiodermatitis, primary biliary cirrhosis, urinary incontinence, chronic cough, asthma, cystitis, and the like.

The cardiovascular disease includes hypertension, blood pressure circadian rhythm abnormality (e.g., early-morning hypertension, nocturnal hypertension etc.), heart diseases (e.g., cardiac hypertrophy, acute heart failure, chronic heart failure including cardiac failure, impaired vasodilation, cardiac myopathy, angina pectoris, myocarditis, atrial fibrillation, arrhythmia, tachycardia, myocardial infarction etc.), cerebrovascular disorders (e.g., asymptomatic cerebrovascular disorder, transient cerebral ischemia, cerebral apoplexy, cerebrovascular dementia, hypertensive encephalopathy, cerebral infarction etc.), cerebral edema, cerebral circulatory disorder, recurrence and sequela of cerebrovascular disorders (e.g., neurotic symptom, psychic symptom, subjective symptom, disorder in daily living activities etc.), ischemic peripheral circulation disorder, myocardial ischemia, venous insufficiency, progression of cardiac insufficiency after myocardial infarction, renal diseases (e.g., nephritis, glomerulonephritis, glomerulosclerosis, renal failure, thrombotic vasculopathy, complication of dialysis, organ dysfunction including nephropathy by radiation damage etc.), arteriosclerosis including atherosclerosis (e.g., aneurysm, coronary sclerosis, cerebral arteriosclerosis, peripheral arterial sclerosis etc.), vascular hypertrophy, vascular hypertrophy or obliteration and organ disorders after intervention (e.g., percutaneous transluminal coronary angioplasty, stenting, coronary angioscopy, intravascular ultrasound, intracoronary thrombolysis etc.), vascular re-obliteration and restenosis after bypass, polycythemia, hypertension, organ disorder and vascular hypertrophy after transplantation, rejection after transplantation, thrombosis, multiple organ disorder, endothelial dysfunction, hypertensive tinnitus, other cardiovascular diseases (e.g., deep vein thrombosis, obstructive peripheral circulatory disorder, arteriosclerosis obliterans, thromboangiitis obliterans, ischemic cerebral circulatory disorder, Raynaud's disease, Buerger's disease etc.), metabolic and/or nutritional disorders (e.g., obesity, hyperlipemia, hypercholesterolemia, hyperuricacidemia, hyperkalemia, hypernatremia etc.), and the like.

The diabetic disease includes diabetes mellitus (e.g., type 1 diabetes mellitus, type 2 diabetes mellitus, gestational diabetes mellitus, obese diabetes mellitus), obesity (e.g., malignant mastocytosis, exogenous obesity, hyperinsulinar obesity, hyperplasmic obesity, hypophyseal adiposity, hypoplasmic obesity, hypothyroid obesity, hypothalamic obesity, symptomatic obesity, infantile obesity, upper body obesity, alimentary obesity, hypogonadal obesity, systemic mastocytosis, simple obesity, central obesity and the like), hyperphagia, hyperlipidemia/dyslipidemia (e.g., hypertriglyceridemia, hypercholesterolemia, high LDL-cholesterolemia, low HDL-cholesterolemia, postprandial hyperlipemia), diabetic complications [e.g., neuropathy, nephropathy, retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, peripheral blood circulation disorder], metabolic syndrome (disease states having 3 or more selected from hypertriglycerid(TG)emia, low HDL cholesterol (HDL-C)emia, hypertension, abdominal obesity and impaired glucose tolerance), and the like.

The digestive organ disease includes an irritable bowel syndrome, inflammatory intestine disease, inflammatory bowel disease, ulcerative colitis, Crohn's disease, diseases caused by a spiral urease-positive gram-negative bacterium (Helicobacter pylori, etc.) (gastritis, gastric ulcer, etc.), gastric cancer, postgastrostomy disorder, indigestion, esophageal ulcer, pancreatitis, polyp of the colon, cholelithiasis, hemorrhoids, peptic ulcer, situational ileitis, gluttony, constipation, diarrhea, borborygmus, non-alcoholic fatty liver disease, visceral pain, gastrointestinal disorder, esophagitis, etc.

The ophthalmologic disease includes terygium, spring catarrh, dry eye, superficial punctate keratopathy and the like.

The cancer disease includes colorectal cancer (e.g. colon cancer, rectal cancer, anal cancer, familial colorectal cancer, hereditary nonpolyposis colorectal cancer and gastrointestinal stromal tumor), lung cancer (e.g. non-small-cell lung cancer, small cell lung cancer and malignant mesothelioma), mesothelioma, pancreatic cancer (e.g. pancreatic ductal carcinoma and pancreatic endocrine tumor), pharyngeal cancer, laryngeal cancer, esophageal cancer, stomach cancer (e.g. papillary adenocarcinoma, mucous adenocarcinoma and adenosquamous carcinoma), duodenal carcinoma, small intestinal cancer, breast cancer (e.g. infiltrating duct carcinoma, noninfiltrating intraductal carcinoma and inflammatory breast cancer), ovarian cancer (e.g. epithelial ovarian cancer, extragonadal germ cell tumor, ovarian germ cell tumor and ovarian low malignant potential tumor), testicular tumor, prostate cancer (e.g. hormone-dependent prostate cancer, hormone-independent prostate cancer and castration-resistant prostate cancer), liver cancer (e.g. hepatic cell carcinoma, primary hepatic cancer and cancer of extrahepatic bile duct), thyroid cancer (e.g. thyroid medullary carcinoma), kidney cancer (e.g. renal cell carcinoma (e.g. clear cell type renal cell carcinoma) and transitional cell carcinoma of renal pelvis and ureter), uterine cancer (e.g. cervical cancer, corpus uteri cancer and uterine sarcoma), gestational choriocarcinoma, brain tumor (e.g. medulloblastoma, glioma, pineal astrocytoma, pilocytic astrocytoma, diffuse astrocytoma, anaplastic astrocytoma and pituitary adenoma), retinoblastoma, skin cancer (e.g. basal cell carcinoma and malignant melanoma), sarcoma (e.g. rhabdomyosarcoma, leiomyosarcoma, soft tissue sarcoma and spindle cell sarcoma), malignant bone tumor, bladder cancer, blood cancer (e.g. multiple myeloma, leukemia (e.g. acute myeloid leukemia), malignant lymphoma, Hodgkin's disease and chronic myeloproliferative disease), and cancer of unknown primary, and the like.

In some embodiments, administering the effective amount of the composition comprising (4-benzyl-4-hydroxypiperidin-1-yl) (2,4′-bipyridin-3-yl)methanone or a pharmaceutically acceptable salt thereof results in (i) a reduction in 24(S)-hydroxycholesterol (24HC) levels and/or (ii) a reduction in NMDA receptor function.

In some embodiments, the effective amount of the composition comprising (4-benzyl-4-hydroxypiperidin-1-yl) (2,4′-bipyridin-3-yl)methanone or a pharmaceutically acceptable salt thereof is administered orally or parenterally (e.g., topically, rectally, intravenously, etc.).

In some embodiments, the effective amount of the composition comprising (4-benzyl-4-hydroxypiperidin-1-yl) (2,4′-bipyridin-3-yl)methanone or a pharmaceutically acceptable salt thereof is administered as a single unit dose. The single unit dose is preferably about 0.01 mg/kg to 100 mg/kg, more preferably about 0.05 mg/kg to 100 mg/kg, still more preferably about 0.1 mg/kg to 10 mg/kg.

In some embodiments, the effective amount of the composition comprising (4-benzyl-4-hydroxypiperidin-1-yl) (2,4′-bipyridin-3-yl)methanone or a pharmaceutically acceptable salt thereof is administered according to a dose regimen.

In some embodiments, the mammal is a human. In further embodiments, the human is an adolescent or an adult.

In some embodiments, the composition comprising (4-benzyl-4-hydroxypiperidin-1-yl) (2,4′-bipyridin-3-yl)methanone or a pharmaceutically acceptable salt thereof further comprises a pharmaceutically acceptable carrier.

In some embodiments, the composition comprising (4-benzyl-4-hydroxypiperidin-1-yl) (2,4′-bipyridin-3-yl)methanone or a pharmaceutically acceptable salt thereof further comprises an additional active agent. Non-limiting examples of additional active agents include: sumatriptan, gabapentin, naratriptan, rizatriptan, ibuprofen, naproxen, dihydroergotamine, amitriptyline, venlafaxine, duloxetine, atenolol, metoprolol, nadolol, propranolol, timolol, propranolol, topiramate, valproate, sodium valproate, erenumab, one-botulinum toxin A (buspirone hydrochloride, tandospirone citrate, osemozotan hydrochloride etc.). Further examples of additional active agents include ketamine, biphosphonates (e.g., risedronate, zoledronate), calcitonin, opioids (e.g., oxycodone, morphine, hydrocodone, fentanyl).

Aspects of this disclosure also relate to a pharmaceutical composition comprising (4-benzyl-4-hydroxypiperidin-1-yl) (2,4′-bipyridin-3-yl)methanone or a pharmaceutically acceptable salt thereof, and a pharmaceutically acceptable carrier, which is for treating pain.

Aspects of this disclosure also relate to (4-benzyl-4-hydroxypiperidin-1-yl) (2,4′-bipyridin-3-yl)methanone or a pharmaceutically acceptable salt thereof for use in treatment of pain.

### DETAILED DESCRIPTION OF THE INVENTION

Aspects of the disclosure relate to a method of treating pain in a mammal (e.g., human, bovine, horse, dog, cat, monkey, mouse, rat; preferably human) comprising administering an effective amount of a composition comprising (4-benzyl-4-hydroxypiperidin-1-yl) (2,4′-bipyridin-3-yl)methanone or a pharmaceutically acceptable salt thereof to the mammal in need thereof. The pain optionally includes inflammatory pain, neuropathic pain, cancer inflammatory pain, post/perioperative pain, and idiopathic pain which is pain of unknown origin, for example, phantom limb pain.

The pain includes preferably Complex Regional Pain Syndrome (CRPS) and migraine, more preferably Complex Regional Pain Syndrome (CRPS).

In addition, aspects of this disclosure relate to a method of treating autoimmune disease, cardiovascular disease, diabetic disease, digestive organ disease, ophthalmologic disease or cancer disease in a mammal comprising administering an effective amount of a composition comprising (4-benzyl-4-hydroxypiperidin-1-yl) (2,4′-bipyridin-3-yl)methanone or a pharmaceutically acceptable salt thereof to the mammal in need thereof.

The autoimmune disease includes rheumatoid arthritis, psoriasis, inflammatory bowel disease (e.g., Crohn's disease, ulcerative colitis etc.), Sjogren's syndrome, Behcet's disease, multiple sclerosis, systemic lupus erythematosus, lupus nephritis, discoid lupus erythematosus, Castleman's disease, ankylopoietic spondylarthritis, polymyositis, dermatomyositis (DM), polyarteritis nodosa (PN), mixed connective tissue disease (MCTD), scleroderma, profundus lupus erythematosus, chronic thyroiditis, Graves' disease, autoimmune gastritis, type I diabetes, autoimmune hemolytic anemia, autoimmune neutropenia, thrombocytopenia, atopic dermatitis, chronic active hepatitis, myasthenia gravis, graft versus host disease, Addison's disease, abnormal immunoresponse, arthritis, dermatitis, radiodermatitis, primary biliary cirrhosis, urinary incontinence, chronic cough, asthma, cystitis, and the like.

The cardiovascular disease includes hypertension, blood pressure circadian rhythm abnormality (e.g., early-morning hypertension, nocturnal hypertension etc.), heart diseases (e.g., cardiac hypertrophy, acute heart failure, chronic heart failure including cardiac failure, impaired vasodilation, cardiac myopathy, angina pectoris, myocarditis, atrial fibrillation, arrhythmia, tachycardia, myocardial infarction etc.), cerebrovascular disorders (e.g., asymptomatic cerebrovascular disorder, transient cerebral ischemia, cerebral apoplexy, cerebrovascular dementia, hypertensive encephalopathy, cerebral infarction etc.), cerebral edema, cerebral circulatory disorder, recurrence and sequela of cerebrovascular disorders (e.g., neurotic symptom, psychic symptom, subjective symptom, disorder in daily living activities etc.), ischemic peripheral circulation disorder, myocardial ischemia, venous insufficiency, progression of cardiac insufficiency after myocardial infarction, renal diseases (e.g., nephritis, glomerulonephritis, glomerulosclerosis, renal failure, thrombotic vasculopathy, complication of dialysis, organ dysfunction including nephropathy by radiation damage etc.), arteriosclerosis including atherosclerosis (e.g., aneurysm, coronary sclerosis, cerebral arteriosclerosis, peripheral arterial sclerosis etc.), vascular hypertrophy, vascular hypertrophy or obliteration and organ disorders after intervention (e.g., percutaneous transluminal coronary angioplasty, stenting, coronary angioscopy, intravascular ultrasound, intracoronary thrombolysis etc.), vascular re-obliteration and restenosis after bypass, polycythemia, hypertension, organ disorder and vascular hypertrophy after transplantation, rejection after transplantation, thrombosis, multiple organ disorder, endothelial dysfunction, hypertensive tinnitus, other cardiovascular diseases (e.g., deep vein thrombosis, obstructive peripheral circulatory disorder, arteriosclerosis obliterans, thromboangiitis obliterans, ischemic cerebral circulatory disorder, Raynaud's disease, Buerger's disease etc.), metabolic and/or nutritional disorders (e.g., obesity, hyperlipemia, hypercholesterolemia, hyperuricacidemia, hyperkalemia, hypernatremia etc.), and the like.

The diabetic disease includes diabetes mellitus (e.g., type 1 diabetes mellitus, type 2 diabetes mellitus, gestational diabetes mellitus, obese diabetes mellitus), obesity (e.g., malignant mastocytosis, exogenous obesity, hyperinsulinar obesity, hyperplasmic obesity, hypophyseal adiposity, hypoplasmic obesity, hypothyroid obesity, hypothalamic obesity, symptomatic obesity, infantile obesity, upper body obesity, alimentary obesity, hypogonadal obesity, systemic mastocytosis, simple obesity, central obesity and the like), hyperphagia, hyperlipidemia/dyslipidemia (e.g., hypertriglyceridemia, hypercholesterolemia, high LDL-cholesterolemia, low HDL-cholesterolemia, postprandial hyperlipemia), diabetic complications [e.g., neuropathy, nephropathy, retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, peripheral blood circulation disorder], metabolic syndrome (disease states having 3 or more selected from hypertriglycerid(TG)emia, low HDL cholesterol(HDL-C)emia, hypertension, abdominal obesity and impaired glucose tolerance), and the like.

The digestive organ disease includes an irritable bowel syndrome, inflammatory intestine disease, inflammatory bowel disease, ulcerative colitis, Crohn's disease, diseases caused by a spiral urease-positive gram-negative bacterium (Helicobacter pylori, etc.) (gastritis, gastric ulcer, etc.), gastric cancer, postgastrostomy disorder, indigestion, esophageal ulcer, pancreatitis, polyp of the colon, cholelithiasis, hemorrhoids, peptic ulcer, situational ileitis, gluttony, constipation, diarrhea, borborygmus, non-alcoholic fatty liver disease, visceral pain, gastrointestinal disorder, esophagitis, etc.

The ophthalmologic disease includes terygium, spring catarrh, dry eye, superficial punctate keratopathy and the like.

The cancer disease includes colorectal cancer (e.g. colon cancer, rectal cancer, anal cancer, familial colorectal cancer, hereditary nonpolyposis colorectal cancer and gastrointestinal stromal tumor), lung cancer (e.g. non-small-cell lung cancer, small cell lung cancer and malignant mesothelioma), mesothelioma, pancreatic cancer (e.g. pancreatic ductal carcinoma and pancreatic endocrine tumor), pharyngeal cancer, laryngeal cancer, esophageal cancer, stomach cancer (e.g. papillary adenocarcinoma, mucous adenocarcinoma and adenosquamous carcinoma), duodenal carcinoma, small intestinal cancer, breast cancer (e.g. infiltrating duct carcinoma, noninfiltrating intraductal carcinoma and inflammatory breast cancer), ovarian cancer (e.g. epithelial ovarian cancer, extragonadal germ cell tumor, ovarian germ cell tumor and ovarian low malignant potential tumor), testicular tumor, prostate cancer (e.g. hormone-dependent prostate cancer, hormone-independent prostate cancer and castration-resistant prostate cancer), liver cancer (e.g. hepatic cell carcinoma, primary hepatic cancer and cancer of extrahepatic bile duct), thyroid cancer (e.g. thyroid medullary carcinoma), kidney cancer (e.g. renal cell carcinoma (e.g. clear cell type renal cell carcinoma) and transitional cell carcinoma of renal pelvis and ureter), uterine cancer (e.g. cervical cancer, corpus uteri cancer and uterine sarcoma), gestational choriocarcinoma, brain tumor (e.g. medulloblastoma, glioma, pineal astrocytoma, pilocytic astrocytoma, diffuse astrocytoma, anaplastic astrocytoma and pituitary adenoma), retinoblastoma, skin cancer (e.g. basal cell carcinoma and malignant melanoma), sarcoma (e.g. rhabdomyosarcoma, leiomyosarcoma, soft tissue sarcoma and spindle cell sarcoma), malignant bone tumor, bladder cancer, blood cancer (e.g. multiple myeloma, leukemia (e.g. acute myeloid leukemia), malignant lymphoma, Hodgkin's disease and chronic myeloproliferative disease), and cancer of unknown primary, and the like.

Not to be bound by theory, it is believed that (4-benzyl-4-hydroxypiperidin-1-yl) (2,4′-bipyridin-3-yl)methanone or a pharmaceutically acceptable salt thereof can reduce NMDA receptor function by blocking the production of 24(S)-hydroxycholesterol (24HC or cerebrosterol), which is the product of CH24H enzymatic activity and a potent (EC50=1.2 μM) and specific positive allosteric modulator of NDMA receptors. By blocking the production of 24(S)-hydroxycholesterol, specific inhibitors of CH24H have the potential to reduce excitatory neurotransmission through NMDA receptors. Accordingly, the CH24H inhibitors disclosed herein are hypothesized to have the same or similar mechanism as ketamine, an NMDA receptor open channel blocker, and, accordingly, confer effects, specifically in a mammal suffering from any one of inflammatory pain, neuropathic pain, cancer inflammatory pain, post/perioperative pain, or idiopathic pain which is pain of unknown origin, for example, phantom limb pain.

Not to be bound by theory, these effects are believed to be dose dependent.

Accordingly, in some embodiments, administering the effective amount of the composition comprising (4-benzyl-4-hydroxypiperidin-1-yl) (2,4′-bipyridin-3-yl)methanone or a pharmaceutically acceptable salt thereof results in (i) a reduction in 24(S)-hydroxycholesterol (24HC) levels and/or (ii) a reduction in NMDA receptor function.

For instance, a reduction in 24(S)-hydroxycholesterol (24HC) levels, recited in (i) above, may be measured by an LC/MS assay. Similarly, a reduction in NMDA receptor function, recited in (ii) above, may be measured electrophysiologically ex-vivo after administration or likely in dissociated neuronal cultures.

In addition to (4-benzyl-4-hydroxypiperidin-1-yl) (2,4′-bipyridin-3-yl)methanone, the CH24H inhibitor is a compound represented by the formula (I):

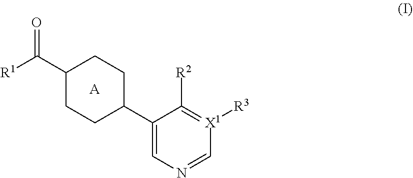

wherein

X¹ is a carbon atom or a nitrogen atom;

Ring A is

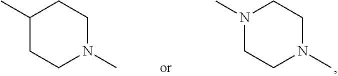

each of which is optionally further substituted and optionally bridged;

R¹ is an optionally substituted C₁₋₆ alkyl group, an optionally substituted hydroxy group, an optionally substituted amino group, an optionally substituted carbocyclic group, or an optionally substituted heterocyclic group, or R″ is optionally bonded to the atom on Ring A to form, together with Ring A, a spiro ring or a fused ring, each of which is substituted by an oxo group and optionally further substituted;

R² is an optionally substituted C₆₋₁₄ aryl group, or an optionally substituted aromatic heterocyclic group; and

R³ is a hydrogen atom or a substituent when X¹ is a carbon atom, or absent when X¹ is a nitrogen atom, (tert-butyl 4-(4-phenylpyrimidin-5-yl)piperazine-1-carboxylate is excluded) or a salt thereof has the same efficacy of (4-benzyl-4-hydroxypiperidin-1-yl) (2,4′-bipyridin-3-yl)methanone.

A variety of compounds represented by formula (I) are provided in US 2015/0315209, the entirety of which is incorporated by reference herein. Further non-limiting examples of a CH24H inhibitor are (2R)-1-((1-(4-(4-methyl-1H-pyrazol-1-yl)pyridin-3-yl)piperidin-4-yl)carbonyl)pyrrolidine-2-carbonitrile and a pharmaceutically acceptable salts thereof. A variety of compounds represented by formula (I) may be a hydrate or a non-hydrate.

### Definitions

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents, and the like to which reference are made in the following description and examples are obtainable from commercial sources, unless otherwise noted.

The following terms are used throughout as defined below.

As used herein, singular articles such as “a” and “an” and “the” and similar referents denote both the singular and the plural, unless otherwise indicated herein or clearly contradicted by context. Disclosure of ranges of values should be understood as a shorthand reference to each separate value falling within the range and at the endpoints, again unless otherwise clearly indicated, and each separate value is to be understood as being described herein as if it were individually set forth.

All methods described herein can be performed in any suitable order unless otherwise indicated herein or otherwise clearly contradicted by context.

The use of any and all examples, or exemplary language (“e.g.” or “such as”) herein, is intended merely to better illuminate the embodiments and does not pose a limitation on the scope of the claims unless otherwise stated. No language in the specification should be construed as indicating any non-claimed feature as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the term “active agent” means a biologically active component of a pharmaceutical composition, e.g. an agent that has a pharmacological effect on the subject or patient to which it is administered. Contemplated herein are (4-benzyl-4-hydroxypiperidin-1-yl) (2,4′-bipyridin-3-yl)methanone or a pharmaceutically acceptable salt thereof and additional active agents for use in combination with (4-benzyl-4-hydroxypiperidin-1-yl) (2,4′-bipyridin-3-yl)methanone or a pharmaceutically acceptable salt thereof. Non-limiting examples of suitable additional active agents for use in combination with the CH24H inhibitors include those mentioned above.

The compound (4-benzyl-4-hydroxypiperidin-1-yl) (2,4′-bipyridin-3-yl)methanone, shown below as Formula (II) is described in U.S. Pat. No. 8,648,079:

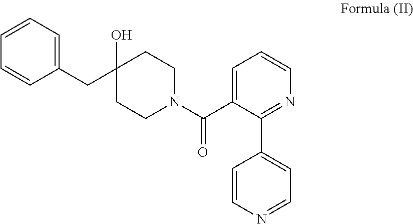 [SMILES: O=C(c1cccnc1-c1ccncc1)N1CCC(O)(Cc2ccccc2)CC1]

The compound of Formula (II) is a CH24H inhibitor. (4-Benzyl-4-hydroxypiperidin-1-yl) (2,4′-bipyridin-3-yl)methanone or a pharmaceutically acceptable salt thereof is safely administered to a mammal, preferably human.

As used herein, the term “effective amount” means an amount effective to successfully achieve a particular biological effect. In the present case, the effective amount is an amount to effective to treat pain which includes inflammatory pain, neuropathic pain, cancer inflammatory pain, post/perioperative pain, and idiopathic pain which is pain of unknown origin, for example, phantom limb pain are included especially.

Neuropathic pain is caused by injury or infection of peripheral sensory nerves. It includes, but is not limited to pain from peripheral nerve trauma, herpes virus infection, diabetes mellitus, causalgia, plexus avulsion, neuroma, limb amputation, and vasculitis.

Neuropathic pain is also caused by nerve damage from chronic alcoholism, human immunodeficiency virus infection, hypothyroidism, uremia, or vitamin deficiencies.

Neuropathic pain includes, but is not limited to pain caused by nerve injury such as, for example, “the pain diabetics suffer from”, burn pain, gout pain, osteoarthritic pain, trigeminal neuralgia pain, acute herpetic and postherpetic pain, causalgia pain, chronic pain, diabetic neuropathic pain, fibromyalgia pain, neuropathic pain associated with diabetic peripheral neuropathy, post herpetic neuralgia, fibromyalgia, neuropathic pain associated with spinal cord injury, Complex Regional Pain Syndrome (CRPS) and migraine. Suitable effective amounts may be determined according to methods well known in the art to determine single unit dosage and/or dose regimens.

In the present another case, the effective amount is an amount to effective to treat autoimmune disease, cardiovascular disease, diabetic disease, digestive organ disease, ophthalmologic disease or cancer disease.

The autoimmune disease includes rheumatoid arthritis, psoriasis, inflammatory bowel disease (e.g., Crohn's disease, ulcerative colitis etc.), Sjogren's syndrome, Behcet's disease, multiple sclerosis, systemic lupus erythematosus, lupus nephritis, discoid lupus erythematosus, Castleman's disease, ankylopoietic spondylarthritis, polymyositis, dermatomyositis (DM), polyarteritis nodosa (PN), mixed connective tissue disease (MCTD), scleroderma, profundus lupus erythematosus, chronic thyroiditis, Graves' disease, autoimmune gastritis, type I diabetes, autoimmune hemolytic anemia, autoimmune neutropenia, thrombocytopenia, atopic dermatitis, chronic active hepatitis, myasthenia gravis, graft versus host disease, Addison's disease, abnormal immunoresponse, arthritis, dermatitis, radiodermatitis, primary biliary cirrhosis, urinary incontinence, chronic cough, asthma, cystitis, and the like.

The cardiovascular disease includes hypertension, blood pressure circadian rhythm abnormality (e.g., early-morning hypertension, nocturnal hypertension etc.), heart diseases (e.g., cardiac hypertrophy, acute heart failure, chronic heart failure including cardiac failure, impaired vasodilation, cardiac myopathy, angina pectoris, myocarditis, atrial fibrillation, arrhythmia, tachycardia, myocardial infarction etc.), cerebrovascular disorders (e.g., asymptomatic cerebrovascular disorder, transient cerebral ischemia, cerebral apoplexy, cerebrovascular dementia, hypertensive encephalopathy, cerebral infarction etc.), cerebral edema, cerebral circulatory disorder, recurrence and sequela of cerebrovascular disorders (e.g., neurotic symptom, psychic symptom, subjective symptom, disorder in daily living activities etc.), ischemic peripheral circulation disorder, myocardial ischemia, venous insufficiency, progression of cardiac insufficiency after myocardial infarction, renal diseases (e.g., nephritis, glomerulonephritis, glomerulosclerosis, renal failure, thrombotic vasculopathy, complication of dialysis, organ dysfunction including nephropathy by radiation damage etc.), arteriosclerosis including atherosclerosis (e.g., aneurysm, coronary sclerosis, cerebral arteriosclerosis, peripheral arterial sclerosis etc.), vascular hypertrophy, vascular hypertrophy or obliteration and organ disorders after intervention (e.g., percutaneous transluminal coronary angioplasty, stenting, coronary angioscopy, intravascular ultrasound, intracoronary thrombolysis etc.), vascular re-obliteration and restenosis after bypass, polycythemia, hypertension, organ disorder and vascular hypertrophy after transplantation, rejection after transplantation, thrombosis, multiple organ disorder, endothelial dysfunction, hypertensive tinnitus, other cardiovascular diseases (e.g., deep vein thrombosis, obstructive peripheral circulatory disorder, arteriosclerosis obliterans, thromboangiitis obliterans, ischemic cerebral circulatory disorder, Raynaud's disease, Buerger's disease etc.), metabolic and/or nutritional disorders (e.g., obesity, hyperlipemia, hypercholesterolemia, hyperuricacidemia, hyperkalemia, hypernatremia etc.), and the like.

The diabetic disease includes diabetes mellitus (e.g., type 1 diabetes mellitus, type 2 diabetes mellitus, gestational diabetes mellitus, obese diabetes mellitus), obesity (e.g., malignant mastocytosis, exogenous obesity, hyperinsulinar obesity, hyperplasmic obesity, hypophyseal adiposity, hypoplasmic obesity, hypothyroid obesity, hypothalamic obesity, symptomatic obesity, infantile obesity, upper body obesity, alimentary obesity, hypogonadal obesity, systemic mastocytosis, simple obesity, central obesity and the like), hyperphagia, hyperlipidemia/dyslipidemia (e.g., hypertriglyceridemia, hypercholesterolemia, high LDL-cholesterolemia, low HDL-cholesterolemia, postprandial hyperlipemia), diabetic complications [e.g., neuropathy, nephropathy, retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, peripheral blood circulation disorder], metabolic syndrome (disease states having 3 or more selected from hypertriglycerid(TG)emia, low HDL cholesterol(HDL-C)emia, hypertension, abdominal obesity and impaired glucose tolerance), and the like.

The digestive organ disease includes an irritable bowel syndrome, inflammatory intestine disease, inflammatory bowel disease, ulcerative colitis, Crohn's disease, diseases caused by a spiral urease-positive gram-negative bacterium (Helicobacter pylori, etc.) (gastritis, gastric ulcer, etc.), gastric cancer, postgastrostomy disorder, indigestion, esophageal ulcer, pancreatitis, polyp of the colon, cholelithiasis, hemorrhoids, peptic ulcer, situational ileitis, gluttony, constipation, diarrhea, borborygmus, non-alcoholic fatty liver disease, visceral pain, gastrointestinal disorder, esophagitis, etc.

The ophthalmologic disease includes terygium, spring catarrh, dry eye, superficial punctate keratopathy and the like.

The cancer disease includes colorectal cancer (e.g. colon cancer, rectal cancer, anal cancer, familial colorectal cancer, hereditary nonpolyposis colorectal cancer and gastrointestinal stromal tumor), lung cancer (e.g. non-small-cell lung cancer, small cell lung cancer and malignant mesothelioma), mesothelioma, pancreatic cancer (e.g. pancreatic ductal carcinoma and pancreatic endocrine tumor), pharyngeal cancer, laryngeal cancer, esophageal cancer, stomach cancer (e.g. papillary adenocarcinoma, mucous adenocarcinoma and adenosquamous carcinoma), duodenal carcinoma, small intestinal cancer, breast cancer (e.g. infiltrating duct carcinoma, noninfiltrating intraductal carcinoma and inflammatory breast cancer), ovarian cancer (e.g. epithelial ovarian cancer, extragonadal germ cell tumor, ovarian germ cell tumor and ovarian low malignant potential tumor), testicular tumor, prostate cancer (e.g. hormone-dependent prostate cancer, hormone-independent prostate cancer and castration-resistant prostate cancer), liver cancer (e.g. hepatic cell carcinoma, primary hepatic cancer and cancer of extrahepatic bile duct), thyroid cancer (e.g. thyroid medullary carcinoma), kidney cancer (e.g. renal cell carcinoma (e.g. clear cell type renal cell carcinoma) and transitional cell carcinoma of renal pelvis and ureter), uterine cancer (e.g. cervical cancer, corpus uteri cancer and uterine sarcoma), gestational choriocarcinoma, brain tumor (e.g. medulloblastoma, glioma, pineal astrocytoma, pilocytic astrocytoma, diffuse astrocytoma, anaplastic astrocytoma and pituitary adenoma), retinoblastoma, skin cancer (e.g. basal cell carcinoma and malignant melanoma), sarcoma (e.g. rhabdomyosarcoma, leiomyosarcoma, soft tissue sarcoma and spindle cell sarcoma), malignant bone tumor, bladder cancer, blood cancer (e.g. multiple myeloma, leukemia (e.g. acute myeloid leukemia), malignant lymphoma, Hodgkin's disease and chronic myeloproliferative disease), and cancer of unknown primary, and the like. Suitable effective amounts may be determined according to methods well known in the art to determine single unit dosage and/or dose regimens.

The term “single unit dose” in this context refers to an effective amount provided in a single administration. Non-limiting examples of suitable single unit doses for use in the claimed methods include about 0.01 to 100 mg/kg body weight, preferably 0.05 to 30 mg/kg body weight, more preferably 0.1 to 10 mg/kg body weight for oral administration to an adult patient (body weight 60 kg).

The term “dose regimen” in this context refers to an effective amount provided over a fixed number of administrations over a specified duration of time.

In some embodiments, the effective amount is administered according to a dose regimen of either twice a day (BID) or once a day (QD) dosing.

It is appreciated that single unit doses may be tailored to the mammal being treated. For example, for humans, non-limiting exemplary single unit doses include less than about 1350 mg, between about 50 mg and about 800 mg (preferably between about 100 mg and about 800 mg), about 100 mg, about 150 mg, about 200 mg, about 300 mg, about 400 mg, about 600 mg or about 800 mg.

Suitable routes of administration and doses and formulations suited thereto are known in the art. Non-limiting examples of routes of administration relevant to the claimed methods include oral and parenteral (e.g., topical, rectal, or intravenous) routes. Examples of the dosage form suited for a particular route of administration include oral preparations such as tablet (including sugar-coated tablet, film-coated tablet, sublingual tablet, orally disintegrating tablet), capsules (including soft capsule, microcapsule), granule, powder, troche, syrup, emulsion, suspension, films (e.g., orally disintegrable films) and the like; and parenteral preparations such as injection (e.g., subcutaneous injection, intravenous injection, intramuscular injection, intraperitoneal injection, drip infusion), external preparations (e.g., dermal preparation, ointment), suppository (e.g., rectal suppository, vaginal suppository), pellet, nasal preparation, pulmonary preparation (inhalant), eye drop and the like. Optionally, these preparations may be a release control preparation (e.g., sustained-release microcapsule) such as an immediate-release preparation or a sustained-release preparation.

The term “pharmaceutically acceptable salt,” as used herein, represents salts or zwitterionic forms of the compounds of the present technology which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present technology can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present technology contemplates sodium, potassium, magnesium, and calcium salts of the compounds of the present technology and the like.

As used herein, the term “pharmaceutically acceptable carrier” means one or more organic or inorganic carrier substances conventionally used in the formulation of pharmaceutical compositions. Suitable pharmaceutically acceptable carriers can be determined by methods well known in the art e.g. excipients, lubricants, binders and disintegrants for solid preparations; solvents, solubilizing agents, suspending agents, isotonicity agents, buffers, and soothing agents for liquid preparations; and/or preparation additives such as preservatives, antioxidants, colorants, and sweetening agents. Non-limiting examples of such suitable pharmaceutically acceptable carriers include:

for an excipient: lactose, sucrose, D-mannitol, D-sorbitol, starch, gelatinated starch, dextrin, crystalline cellulose, low-substituted hydroxypropylcellulose, sodium carboxymethylcellulose, gum arabic, pullulan, light anhydrous silicic acid, synthesis aluminum silicate and magnesium alumino metasilicate;

for a lubricant: magnesium stearate, calcium stearate, talc and colloidal silica;

for a binder: gelatinated starch, sucrose, gelatin, gum arabic, methylcellulose, carboxymethylcellulose, sodium carboxymethylcellulose, crystalline cellulose, sucrose, D-mannitol, trehalose, dextrin, pullulan, hydroxypropylcellulose, hydroxypropylmethylcellulose and polyvinylpyrrolidone;

for a disintegrant: lactose, sucrose, starch, carboxymethylcellulose, calcium carboxymethylcellulose, croscarmellose sodium, sodium carboxymethyl starch, light anhydrous silicic acid and low-substituted hydroxypropylcellulose;

for a solvent: water, physiological brine, Ringer's solution, alcohol, propylene glycol, polyethylene glycol, sesame oil, corn oil, olive oil and cottonseed oil;

for a solubilizing agent: polyethylene glycol, propylene glycol, D-mannitol, trehalose, benzyl benzoate, ethanol, trisaminomethane, cholesterol, triethanolamine, sodium carbonate, sodium citrate, sodium salicylate and sodium acetate;

for a suspending agent: surfactants such as stearyltriethanolamine, sodium lauryl sulfate, lauryl aminopropionate, lecithin, benzalkonium chloride, benzethonium chloride, and glycerol monostearate; hydrophilic polymers such as polyvinyl alcohol, polyvinylpyrrolidone, sodium carboxymethylcellulose, methylcellulose, hydroxymethylcellulose, hydroxyethylcellulose, hydroxypropylcellulose and the like; polysorbates, and polyoxyethylene hydrogenated castor oil;

for an isotonicity agent: sodium chloride, glycerol, D-mannitol, D-sorbitol and glucose;

for a buffer: phosphate, acetate, carbonate, and citrate;

for a soothing agent: benzyl alcohol;

for a preservative: p-oxybenzoates, chlorobutanol, benzyl alcohol, phenethyl alcohol, dehydroacetic acid, and sorbic acid;

for an antioxidant: sulfite and ascorbate;

for a colorant: aqueous water-soluble food tar colors (e.g., food colors such as Food Color Red Nos. 2 and 3, Food Color Yellow Nos. 4 and 5, Food Color Blue Nos. 1 and 2 and the like), water insoluble lake dyes (e.g., aluminum salt of the above-mentioned water-soluble food tar color), and natural dyes (e.g., β-carotene, chlorophyll, ferric oxide red); and

for a sweetening agent: saccharin sodium, dipotassium glycyrrhizinate, aspartame, and stevia.

As used herein, the term “treating” includes the prevention, reduction, and/or complete resolution of the symptoms associated with or the cause of the target indication and/or a lessening of severity of the condition.

In some embodiments, (4-benzyl-4-hydroxypiperidin-1-yl) (2,4′-bipyridin-3-yl)methanone or a pharmaceutically acceptable salt thereof may be used in combination with other therapies including spinal code stimulators, sympathectomy and amputation.

### EXAMPLES

The present invention is explained in detail in the following by referring to Examples, which are not to be construed as limitative, and the invention may be changed within the scope of the present invention.

### Example 1—Efficacy Data in Preclinical CRPS Model

The following Example provides data from a mouse model of chronic post-ischemic pain, which recapitulates symptoms seen in CRPS patients with peripheral tissue injury and subsequent progression of allodynia. (4-Benzyl-4-hydroxypiperidin-1-yl) (2,4′-bipyridin-3-yl)methanone was tested in the CRPS model for its potential effects on pain. Following 3-hour ischemia and 0.5-hour reperfusion given at a hind limb, mice (C57) were orally treated with (4-benzyl-4-hydroxypiperidin-1-yl) (2,4′-bipyridin-3-yl)methanone at the dose of 30 mg/kg once daily for 7 days (n=8). Paw withdraw threshold was evaluated with von Frey filaments as a behavioral index of mechanical allodynia at 0 h for the base line, 6 h, Days 1, 2, 4 and 7. The cumulative score of paw withdraw threshold over the study period was 17.7 in the sham-treated control group without ischemia/reperfusion injury. In the groups given ischemic injury, the cumulative score was 9.0 and 16.2 for the vehicle control and (4-benzyl-4-hydroxypiperidin-1-yl) (2,4′-bipyridin-3-yl)methanone group, respectively. It was hence indicated that allodynia threshold was markedly increased by treatment with (4-benzyl-4-hydroxypiperidin-1-yl) (2,4′-bipyridin-3-yl)methanone. These results support the use of (4-benzyl-4-hydroxypiperidin-1-yl) (2,4′-bipyridin-3-yl)methanone to treat the pain sensitization in CRPS.

### Example 2—Efficacy Data in Preclinical Migraine Model

The following Example provides data from a rat cortical spreading depression (CSD) model, which recapitulates symptoms seen in migraine patients with aura. (2R)-1-((1-(4-(4-Methyl-1H-pyrazol-1-yl)pyridin-3-yl)piperidin-4-yl)carbonyl)pyrrolidine-2-carbonitrile was tested in the CSD model for its potential effects on hyperperfusion events and cortical direct current (DC)-potentials. Adult female Wistar rats from Charles River Germany were used for the experiment. Animals were housed at a standard temperature (22±1° C.) and in a light-controlled environment (lights on from 7 am to 8 pm) with ad libitum access to food and water. Rats were orally treated with vehicle (0.5% methylcellulose in water) or (2R)-1-((1-(4-(4-methyl-1H-pyrazol-1-yl)pyridin-3-yl)piperidin-4-yl)carbonyl)pyrrolidine-2-carbonitrile at the dose of 10 mg/kg once daily for 6 days (n=8). One hour after the last dosing, rats were anesthetized with 5% isoflurane (in 70% N₂O and 30% O₂; flow 300 ml/min), and placed in a stereotactic frame.

During the operation and CSD the concentration of anesthetic was reduced to 1-1.5%. The rectal temperature was maintained at 37.0±1.0° C. with a homeothermic blanket system. The skin was opened by a medial incision and retracted laterally. Three burr holes were drilled under saline cooling over the right hemisphere at the following coordinates (mm from bregma): (1) posterior 4.5, lateral, 2.0 (occipital cortex): KCl application site; (2) posterior 0.5, lateral 2.0 (parietal cortex) (3) anterior 2, lateral 2 (frontal cortex). A laser-Doppler flow probe (Oxyflow, Oxford Optronics, UK) to monitor cerebral blood flow (CBF) was placed in the parietal cortex burr holes on the intact dura. An invasive Ag/AgCl electrode for measuring DC potential shifts was placed in the frontal cortex burr holes on the intact dura. For the DC-potential measurement, a reference electrode was fixed in the neck. After surgical preparation, the cortex was allowed to recover for 15 minutes under saline irrigation. One molar KCl was placed on the pial surface and kept moist by placing 5 μl of KCl solution every 15 minutes. The number of KCl-induced CSDs was checked over the period of 2 hours and the number of CBF changes was also measured. The average number of KCl-induced CSDs was 10.00 and 4.75 for the vehicle-treated group and the (2R)-1-((1-(4-(4-methyl-1H-pyrazol-1-yl)pyridin-3-yl)piperidin-4-yl)carbonyl)pyrrolidine-2-carbonitrile-treated group, respectively. The average number of CBF changes was 22.75 and 15.75 for the vehicle-treated group and the (2R)-1-((1-(4-(4-methyl-1H-pyrazol-1-yl)pyridin-3-yl)piperidin-4-yl)carbonyl)pyrrolidine-2-carbonitrile-treated group, respectively. It was hence indicated that CSD events were markedly reduced by treatment with (2R)-1-((1-(4-(4-methyl-1H-pyrazol-1-yl)pyridin-3-yl)piperidin-4-yl)carbonyl)pyrrolidine-2-carbonitrile. These results support the use of (2R)-1-((1-(4-(4-methyl-1H-pyrazol-1-yl)pyridin-3-yl)piperidin-4-yl)carbonyl)pyrrolidine-2-carbonitrile to treat migraine.

The efficacy of (4-benzyl-4-hydroxypiperidin-1-yl) (2,4′-bipyridin-3-yl)methanone in the treatment of migraine may be confirmed in the same manner as in Example 2.

### Example 3—Efficacy, Safety and Tolerability of (4-benzyl-4-hydroxypiperidin-1-yl)(2,4′-bipyridin-3-yl)methanone as an Adjunctive Therapy in Adult Subjects with Chronic Complex Regional Pain Syndrome

This is a randomized, double-blind, placebo-controlled, parallel-group study in adult subjects (18 years) with chronic (symptoms ≥6 months) CRPS. The objective will be to look for a signal to determine if (4-benzyl-4-hydroxypiperidin-1-yl) (2,4′-bipyridin-3-yl)methanone as adjunctive therapy can reduce pain as measured by the NPS (an 11-point scale by electronic pain diary). This study will also evaluate efficacy of (4-benzyl-4-hydroxypiperidin-1-yl) (2,4′-bipyridin-3-yl)methanone as measured by PROMIS-29 version 2, PGIC, and CSS.

Approximately 24 subjects will be randomized to ensure 20 completers in the double-blind phase of the study.

Randomization will be 2:1 (treatment:placebo).

This study consists of 2 parts:

### Part A: Double-Blind Treatment

2- to 4-week screening/baseline period.
3-week titration period.
4-week maintenance period.
Follow-up/taper period if not rolling over into Part B

### Part B: Open-Label Extension

12-week open label.
Follow-up/taper period.

### Part A: Double-Blind Treatment (9-13 Weeks)

At the screening visit (Visit 1), after obtaining informed consent, subjects will undergo screening procedures to assess subject eligibility in accordance with study entry criteria. Subjects who fulfill the CRPS Budapest Criteria, and have symptoms for 6 months and meet inclusion/exclusion criteria at the screening/baseline visit will be eligible for entry into the study. For a minimum of 6 of the last 7 screening days prior to enrollment into the study, baseline current pain intensity will be collected 3 times a day to provide an average daily 24-hour pain intensity (NPS; an 11-point scale by electronic pain diary). The baseline will be defined as the mean of the average screening 24-hour pain intensity score for the last 7 days prior to the first dose. During the Part A, average 24-hour pain intensity will be collected 3 times a day. Pain intensity score collected during the last 7 days prior to Day 14 and Day 49 (or the last dose in Part A) will be used as to calculate the average 24-hour pain intensity score for the primary endpoint analysis.

At the end of the prospective screening/baseline period, subjects will return to the clinic (Visit 2, Day 1) and if a subject does not meet the eligibility criteria the subject will be discontinued from the study and considered a screen failure.

On Day 1, the subjects who meet the entry criteria will be randomized in a 2:1 ratio to double-blind treatment with investigational product (IP), either (4-benzyl-4-hydroxypiperidin-1-yl) (2,4′-bipyridin-3-yl)methanone 100 mg tablets or matching placebo, for 7 weeks (3-week titration period and 4-week maintenance period).

All screening/baseline assessments will be collected prior to initiating treatment.

At Visit 2 (Day 1), after randomization and all predose procedures have been performed, subjects will be started on 100 mg BID IP (either (4-benzyl-4-hydroxypiperidin-1-yl) (2,4′-bipyridin-3-yl)methanone 100 mg tablets or matching placebo) for approximately 1 week. The site will contact the subject to determine safety and tolerability and the dose will be increased 200 mg BID IP for approximately 1 week.

At Visit 3, safety and tolerability will be assessed and if the drug is well tolerated and the subject continues to need a higher dose, the dose will be increased to 300 mg BID. After a week at 300 mg BID, the site will contact the subject to determine safety and tolerability of this dose. After completing 1 week at 300 mg BID, the 3-week titration period will be completed and the 4-week maintenance period will begin.

If at any time during the 3-week titration period the subject cannot tolerate the dose, the dose may be decreased to the previous tolerated dose. If the subject cannot tolerate the minimum daily dose of 100 mg BID, the subject will be withdrawn from the study. If the dose is decreased due to tolerance, based on the investigator's review, the dose may be increased to the next highest dose 1 time during the titration period. Dose modifications/up-titrations outside of this period should be discussed with the medical monitor.

The dose at end of the titration period will be continued through the maintenance period. During the 4-week maintenance period, nonemergency IP dose changes should be discussed with the sponsor/medical monitor prior to initiation. The subject should be instructed not to alter their dose without prior approval from the investigator. Any change in dose will be documented in the subject's clinic chart and dosing card.

Pain medications and nondrug treatments must be stable (regimented per prescription) for 1 month prior to screening and should remain stable throughout Part A. Pain medication use may be adjusted under supervision during Part B. Concurrent treatment regimen data will be collected throughout the study.

A single effective rescue medication must be identified for each subject for use during the study.

The prescribed maximum dose must remain stable during Part A. The use of rescue pain medications will be assessed at each visit; subjects requiring significant increase of rescue medication (frequency or dose 50% over pre-enrollment levels or over the prescribed maximum) during Part A will be considered for withdrawal from the study at the investigator's discretion.

During Part A, all subjects will continue to enter the current pain intensity score as described 3 times a day using the NPS in the electronic pain diary. This data will be captured daily during Part A.

An unblinded interim analysis will be conducted when all subjects have completed the double-blind Part A.

Following completion of Part A, subjects will have the option to continue into Part B, a 12-week open-label extension study part, or to enter a double-blind taper period (maximum 6 days).

In all subjects choosing the double-blind taper period, the IP dose will be reduced to the next lower dose every 3 days (or less frequently based on the investigator's discretion) until IP dose is discontinued. After tapering, the subjects will complete a safety follow-up phone call approximately 15 days after the last dose of IP.

### Part B Open-Label Extension (12 Weeks):

Since the therapy assignment will remain blinded, all subjects who choose to continue into Part B will start at 200 mg BID (4-benzyl-4-hydroxypiperidin-1-yl) (2,4′-bipyridin-3-yl)methanone (100 mg tablets), regardless of the treatment they were on in Part A. Subjects should remain at this dose for 48 hours after which the dose may be increased or decreased based on tolerability. The subject should be instructed not to alter their dose without prior approval from the investigator. Any change in dose will be documented in the subject's clinic chart and dosing card. Subjects will visit the clinic approximately every 6 weeks. The current pain intensity will be collected 3 times a day using the electronic pain diary. From this data an average 24-hour pain intensity score can be calculated. In addition, using the data collected in the last 7 days prior to Day 91 and Day 133 (or the last dose in Part B), the average pain score prior to these visits will be derived.

During the taper period, the IP dose will be reduced to the next lower dose every 3 days (or less frequently based on the investigator's discretion) until IP dose is discontinued. After tapering, the subjects will complete a safety follow-up phone call approximately 15 days after the last dose of IP and exit the study.

The total study duration from screening to the last visit in Part B will be approximately 6 months.

### Example 4—a Randomized, Double-Blind, Placebo-Controlled, Parallel-Group Study to Evaluate the Efficacy, Tolerability and Safety of (4-benzyl-4-hydroxypiperidin-1-yl)(2,4′-bipyridin-3-yl)methanone as Monotherapy for Prophylaxis in Adult Patients with Episodic Migraine with and/or without Aura

This is a randomized, double-blind, placebo-controlled, parallel-group study in adult patients (18-65 years, inclusive) with episodic migraine with and/or without aura for at least 1 year prior to screening (as per ICHD-3 criteria) and with onset prior to age 50. The primary objective will be to determine if (4-benzyl-4-hydroxypiperidin-1-yl) (2,4′-bipyridin-3-yl)methanone, as monotherapy, can reduce the frequency of migraine headache days per 28 days. The study will evaluate efficacy as the change from baseline in migraine headache days as recorded in headache diary. In addition, the study will evaluate the potential improvement after treatment with (4-benzyl-4-hydroxypiperidin-1-yl) (2,4′-bipyridin-3-yl)methanone in the frequency of the headache days, in the intensity of migraine headaches and headaches overall, and in the Patient Global Impression of Severity (PGI-S).

Approximately 40 patients will be randomized. Randomization will be 1:1 (20 treatment:20 placebo).

This study consists of 2 parts:

### Part A: Double-Blind

4-week Screening/Baseline Period
3-week Titration Period
12-week Maintenance Period
1-week Down-Titration Period if not rolling over into part B

### Part B: Open-Label Extension

12-week open label
1-week Down-Titration Period

### Part A: Double-Blind Part (19-20 Weeks)

At the Screening Visit (Visit 1), after obtaining informed consent, patients will undergo screening procedures to assess patient eligibility in accordance with study entry criteria. Patients who have symptoms for at least 1 year and meet all inclusion/exclusion criteria at the Screening/Baseline Visit will be eligible for entry into the study.

At the end of the prospective Screening/Baseline Period, patients will return to the clinic (Visit 2, Day 1). Patients who do not meet the eligibility criteria or did not comply with the data collection will be discontinued from the study and considered screen failure.

On Day 1, the patients who meet the entry criteria will be randomized in a 1:1 ratio to double-blind treatment with Investigational Product (IP), either (4-benzyl-4-hydroxypiperidin-1-yl) (2,4′-bipyridin-3-yl)methanone or matching placebo for 15 weeks (3-week Titration Period, 12-week Maintenance Period). All Screening/Baseline assessments will be collected prior to initiating treatment.

At Visit 2 (Day 1), after randomization and all pre-dose procedures have been performed, patients will be started on 100 mg BID Investigational Product (IP) (either (4-benzyl-4-hydroxypiperidin-1-yl) (2,4′-bipyridin-3-yl)methanone or identical placebo) for 1 week. The site will contact the patient via phone to determine safety and tolerability (Visit 3, Day 7) and the dose will be increased to 200 mg BID IP for one week. At Visit 4 (Day 14), risk/benefit will be assessed and if the drug is well tolerated, the dose will be increased to 300 mg BID. After a week at 300 mg BID, the site will contact the patient via phone to determine safety and tolerability of this dose (Visit 5, Day 21). After one-week dosing at 300 mg BID, the 3-week Titration Period will be completed, and the 12-week Maintenance Period will begin. If at any time during the 3-week Titration Period the patient cannot tolerate the dose, the dose may be decreased to the previous tolerated dose. If the patient cannot tolerate the minimum daily dose of 100 mg BID, the patient will be withdrawn from the study. If the dose has been decreased due to tolerance, based on the Investigator's review, the dose may be increased to the next highest dose one time during the Titration Period. The IP dose selected at end of titration period will be continued through maintenance period. During the 12-week Maintenance Period, IP non-emergency dose changes should be discussed with the Sponsor/Medical Monitor prior to initiation. The subject should be instructed not to alter their dose without prior approval from the investigator.

At Weeks 7 and 14, sites will contact patients by phone to monitor study drug compliance, concomitant medication use changes, AEs, and provide instructions regarding continued dosing. Any change in dose will be documented in the patient's source doc and dosing card.

Migraine Specific Medications (MSM, rescue medication only), and non-drug treatments must be stable for 3 months prior to enrollment (screening) and not be altered during the treatment period. Concurrent treatment regimen data will be collected throughout the study.

An effective MSM must be identified for each patient for use during the trial. The prescribed maximum dose for the rescue medication must remain stable during part A. The use of MSM will be assessed at visits; patients requiring significant increase of MSM (frequency or dose 50% over pre-screening levels or over the prescribed maximum) will be considered for withdrawal from the study at the Investigator discretion.

All patients will continue to log each occurring headache, including intensity, and aura characteristics (if any) daily and will follow-up with in-person visits on 6-week intervals (Visit 6, Day 63 and Visit 7, Day 105).

Following completion of part A, subjects will have the option to continue into part B, a 12-week, open-label drug extension or to enter a 1-week double-blind taper period.

In all subjects choosing the taper period, the IP dose will be reduced to the next lower dose every 3 days (or less frequently based on the investigator's discretion) until IP dose is discontinued. After tapering, the subjects will complete a safety follow-up phone call approximately 14 days after the last dose of IP.

### Part B Open-Label Extension (12 Weeks):

Since the treatment assignments will remain blinded, all subjects who choose to continue into part B will start at 200 mg BID (4-benzyl-4-hydroxypiperidin-1-yl) (2,4′-bipyridin-3-yl)methanone regardless of the treatment they were on in part A. Subjects should remain at this dose for 48 hours after which the dose may be increased to 300 mg BID or decreased to 100 mg BID based on tolerability via phone based on investigator discretion. Subjects will follow-up with in-person visits on 6-week intervals (Visit 8, Day 147 and Visit 9, Day 189). The subject should be instructed not to alter their dose without prior approval from the investigator. Any change in dose will be documented in the subject's clinic chart and dosing card. All patients will continue to log each occurring headache, including intensity and aura characteristics (if any), daily.

During the taper period, the IP dose will be reduced to the next lower dose every 3 days (or less frequently based on the investigator's discretion) until IP dose is discontinued. After tapering, the subjects will complete a safety follow-up phone call approximately 14 days after the last dose of IP and exit the study.

The total study duration to the last visit will be approximately 8 months.

### Procedures:

Scales: The migraine headache and headache frequency, intensity and aura characteristics (if any) will be measured with a headache diary daily from Visit 1 up to Visit 9. The PGI-S will be administered at Visits 2, 6, 7 and 9.
Laboratory Sampling: Blood samples for clinical safety laboratory tests (5 mL at each sample time point) including hematology and chemistry, will be collected at Visit 1 (Screening/Baseline), Visit 2, and Visit 6. Blood Samples for population PK analysis of (4-benzyl-4-hydroxypiperidin-1-yl) (2,4′-bipyridin-3-yl)methanone will be drawn at Visit 2, Visit 6 and Visit 7. Blood draws for the biomarker 24HC will be drawn at Visit 1 (Baseline), Visit 2 and Visit 6. Blood for pregnancy testing will be collected for all females of childbearing potential at Visit 1 (Screening/Baseline) and Visit 6 and urine pregnancy testing will be collected for all females of childbearing potential at Visit 2 and Visit 9. In addition, urinalysis for urine for drug (illicit) screening will be performed at Visit 1 (Screening//Baseline). Cannabis use will be allowed in the study.
Electrocardiograms: Echocardiograms will be administered at Visit 1, Visit 6, and Visit 8.

### Formulation Example

For (4-benzyl-4-hydroxypiperidin-1-yl) (2,4′-bipyridin-3-yl)methanone, the formulation (film coating tablet) was produced in line with the following specification (Table 1).

TABLE 1

| | | Quantity per |
|---|---|---|
| | | Tablet(mg) |
| | Components | 20 mg |
| Core | (4-benzyl-4-hydroxypiperidin-1- | 20 |
| Tablet | yl)(2,4′-bipyridin-3-yl)methanone | |
| (Internal | Microcrystalline cellulose | 1.5 |
| granules) | (PH101) | |
| | Low-Substituted Hydroxypropyl | 1.25 |
| | Cellulose (L-HPC 21) | |
| | Hydroxypropyl Cellulose | 0.75 |
| (External | Low-Substituted Hydroxypropyl | 1.25 |
| granules) | Cellulose (L-HPC 21) | |
| | Magnesium Stearate | 0.25 |
| Coating | (OPADRY Red 03F45081) | 0.508 |
| Solution | (OPADRY Yellow 03F42240) | 0.508 |
| | Hypromellose 2910¹⁾ | (0.75) |
| | Polyethylene Glycol 8000¹⁾ | (0.167) |
| | Titanium Dioxide¹⁾ | (0.083) |
| | Ferric Oxide, Red¹⁾ | (0.008) |
| | Ferric Oxide, Yellow¹⁾ | (0.008) |
| | Total | 26.016 |

1) These ingredients are components of OPADRY® Red 03F45081 and OPADRY (registered trademark) Yellow 03F42240 (premixed coating materials).

The following references are incorporated by reference herein to the same extent as if individually incorporated.
1. Johanna C. M. Schilder. et al. Pain Relief Is Associated With Improvement in Motor Function in Complex Regional Pain Syndrome Type 1: Secondary Analysis of a Placebo-Controlled Study on the Effects of Ketamine. The Journal of Pain Vol 14, No. 11 (November) 2013: 1514-1521
2. Robert J Schwartzman. et al. The use of ketamine in complex regional pain syndrome: possible mechanisms. Expert Rev. Neurother. 11(5), 719-734 (2011)
3. En Lin Goh. et al. Complex regional pain syndrome: a recent update. Burns & Trauma (2017) 5: 2
4. O'Connell N E. et al. Interventions for treating pain and disability in adults with complex regional pain syndrome—an overview of systematic reviews (Review). Cochrane Database of Systematic Reviews 2013, Issue 4. Art. No.: CD9416
5. David W. Dodick. et al. Effect of Fremanezumab Compared With Placebo for Prevention of Episodic Migraine. JAMA, 2018; 319(19): 1999-2008
6. Virginia L. Stauffer. et al. Evaluation of Galcanezumab for the Prevention of Episodic Migraine. JAMA Neurol. doi: 10.1001/jamaneurol.2018.1212

This application is based on U.S. patent application No. 62/733,927 filed on Sep. 20, 2018 in USA, the contents of which are encompassed in full herein.

1. A method of treating pain in a mammal comprising administering an effective amount of a composition comprising (4-benzyl-4-hydroxypiperidin-1-yl) (2,4′-bipyridin-3-yl)methanone or a pharmaceutically acceptable salt thereof to the mammal in need thereof.

2. The method of claim 1, wherein the pain is Complex Regional Pain Syndrome (CRPS).

3. The method of claim 1, wherein the pain is migraine.

4. The method of claim 1, wherein administering the effective amount of the composition comprising (4-benzyl-4-hydroxypiperidin-1-yl) (2,4′-bipyridin-3-yl)methanone or a pharmaceutically acceptable salt thereof results in (i) a reduction in 24(S)-hydroxycholesterol (24HC) levels and/or (ii) a reduction in NMDA receptor function.

5. The method of claim 1, wherein the effective amount of the composition comprising (4-benzyl-4-hydroxypiperidin-1-yl) (2,4′-bipyridin-3-yl)methanone or a pharmaceutically acceptable salt thereof is administered orally or parenterally.

6. The method of claim 1, wherein the effective amount of the composition comprising (4-benzyl-4-hydroxypiperidin-1-yl) (2,4′-bipyridin-3-yl)methanone or a pharmaceutically acceptable salt thereof is administered as a single unit dose.

7. The method of claim 6, wherein the single unit dose is about 0.01 mg/kg to 100 mg/kg.

8. The method of claim 6, wherein the single unit dose is about 0.05 mg/kg to 100 mg/kg.

9. The method of claim 6, wherein the single unit dose is about 0.1 mg/kg to 10 mg/kg.

10. The method of claim 1, wherein the effective amount of the composition comprising (4-benzyl-4-hydroxypiperidin-1-yl) (2,4′-bipyridin-3-yl)methanone or a pharmaceutically acceptable salt thereof is administered according to a dose regimen.

11. The method of claim 1, wherein the mammal is a human.

12. The method of claim 1, wherein the composition comprising (4-benzyl-4-hydroxypiperidin-1-yl) (2,4′-bipyridin-3-yl)methanone or a pharmaceutically acceptable salt thereof further comprises a pharmaceutically acceptable carrier.

13. The method of claim 1, wherein the composition comprising (4-benzyl-4-hydroxypiperidin-1-yl) (2,4′-bipyridin-3-yl)methanone or a pharmaceutically acceptable salt thereof further comprises an additional active agent.

14. A pharmaceutical composition comprising (4-benzyl-4-hydroxypiperidin-1-yl) (2,4′-bipyridin-3-yl)methanone or a pharmaceutically acceptable salt thereof, and a pharmaceutically acceptable carrier, which is for treating pain.

15. (4-Benzyl-4-hydroxypiperidin-1-yl) (2,4′-bipyridin-3-yl)methanone or a pharmaceutically acceptable salt thereof for use in treatment of pain.
